(19) United States
(12) Patent Application Publication
(10) Pub. No.: US 2022/0133282 A1
(43) Pub. Date: May 5, 2022

(54) SECURE DEVICE FOR DELIVERING MEDICATIONS

(71) Applicant: Achilleus LLC, Kansas City, MO (US)

(72) Inventor: Brian Sexton, Chicago, IL (US)

(21) Appl. No.: 17/644,327

(22) Filed: Dec. 14, 2021

Related U.S. Application Data
(63) Continuation-in-part of application No. 16/588,947, filed on Sep. 30, 2019.
(60) Provisional application No. 62/738,699, filed on Sep. 28, 2018.
(60) Provisional application No. 63/199,210, filed on Dec. 14, 2020.

(51) Int. Cl. A61B 10/00; A61B 5/01; A61B 5/00; G16H 20/10

(52) U.S. Cl. CPC A61B 10/0045; A61B 5/01; G16H 20/10; A61B 5/74; A61B 5/6802

(57) ABSTRACT

A device for delivering medications that includes a syringe body, a cartridge nest with an application tip, a plunger actuator, an electronic subassembly adapted for collecting vital signs information, and a biological fluid specimen collector adapted for collecting a specimen from a patient. The biological fluid specimen collector can be adapted for nasopharyngeal or oropharyngeal sampling, and may be an absorbent material located at least partially around an application injector extending from the application tip. The electronic subassembly may be configured for interfacing with one or more peripheral sensors, transmitting vital signs information to a database, and transmitting a notification when at least one vital sign parameter meets a predetermined threshold.

### CROSS-REFERENCE TO RELATED APPLICATIONS

This application is a continuation-in-part of and claims priority to U.S. patent application Ser. No. 16/588,947 filed Sep. 30, 2019, to Brian Sexton, entitled “Secure Device for Delivering Medications,” currently pending, which claims priority to U.S. Provisional Patent Application No. 62/738,699, filed on Sep. 28, 2018, to Brian Sexton, entitled “Secure Device for Delivering Intranasal Medication.′ This application also claims priority to U.S. Provisional Patent Application Ser. No. 63/199,210, filed on Dec. 14, 2020, to Brian Sexton, entitled “Secure Device for Delivering Medications,” currently pending. The entire disclosures, including the specifications and drawings, of all the above-referenced applications are incorporated herein by reference.

### FIELD OF THE INVENTION

The present invention is generally directed to a system and method for delivering medications and to a system and method of monitoring health information.

### BACKGROUND

Intranasal medications may be delivered by way of an injector through a nasal passage of a patient and into a region medial, posterior, and/or inferior to a sphenopalatine ganglion of the patient. Currently known devices generally provide comfort and convenience for the patient and the administrator, and are often used for administering pain medications, among other treatments. Currently known, generally unsecure, devices risk being abused and misused. Existing devices are contributing to an ongoing opioid epidemic in the United States and elsewhere, and risk untraceable applications of vaccines and pain medications. Further, when administering medications, a patient's vitals and health metrics are often measured and recorded. However, with known methods, this typically involves the use of multiple devices. It is also desirable for such health metrics and vitals to be recorded and possibly transmitted. Accordingly, there is a need for a device that can prevent misuse and abuse of pain medications, while allowing accurate and convenient control for medical professionals. There is a further need for a single device that is capable of administering medications, collecting and transmitting a patient's health information, and receiving data from external sources.

### SUMMARY OF THE INVENTION

One embodiment of the present invention generally relates to a syringe or secure device that may be adapted for requiring a form of verification and approval prior to the administration of medication, to help curb, reduce, or eliminate medication abuse or misuse. Individual medications can be contained within a medicine cartridge, which may in turn be outfitted with a unique identifier. In one embodiment, this medicine cartridge may remain locked and unusable unless it is paired with a compatible syringe body. The medicine cartridge may contain information such as, approved administrator(s) (e.g., doctor, nurse, patient, etc.), medicine type, dose, pre-approved syringe identification, among other information.

The medicine cartridge can be loaded, placed, or otherwise secured within a cartridge nest prior to the administration of the medication. The cartridge nest may further house an application tip that navigates through a patient's nasal cavity to reach the desired location within the sinuses, when it is desirable to administer medication to the patient's nasal cavity.

The cartridge nest may be attached or otherwise secured to a syringe body to further assist in administering medication to the patient. The syringe body can contain an additional electronic subassembly, which may aid in securing the device to prevent abuse or misuse, as well as acting as a central hub for housing additional electronic or medical components. In one embodiment, the electronic subassembly may lock the device and prevent the administration of medication unless authentication information is provided which unlocks the device. The information may contain, for example: pre-approved syringe identification, approved administrators, time stamp of application, location of application, approved medicines, approved doses, and Blockchain methodology for auditing.

In one embodiment, the electronic subassembly may further comprise an apparatus for taking a patient's temperature, which may be an infrared proximity temperature, and an optional communication module associated therewith.

The secure device may further be compatible with a dedicated software application, in which each individual user or patient may have their own unique profile. The dedicated software application may be used to provide both the authentication approval for using the secure device, as well as for storing health metrics and other health information of the user. Further, the dedicated software application may be used to connect and share information between multiple users, healthcare providers and/or other organizations. Additionally, active scanning of electronic devices containing the dedicated software application may be used to generate a physical proximity network, cataloging the location and proximity of multiple users. Such physical proximity network may be particularly beneficial in contact tracing and notifying users of potential exposure to another user that has tested positive for a transmittable disease.

Other and further objects of the invention, together with the features of novelty appurtenant thereto, will appear in the course of the following description.

### BRIEF DESCRIPTION OF THE SEVERAL VIEWS OF THE DRAWING

In the accompanying drawings, which form a part of the specification and are to be read in conjunction therewith in which like reference numerals are used to indicate like or similar parts in the various views:

FIG. 1 is a perspective view of a secure syringe in accordance with one embodiment of the present invention;

FIG. 1A is a perspective view of a secure syringe in accordance with one embodiment of the present invention;

FIG. 2 is an exploded perspective view of a secure syringe in accordance with one embodiment of the present invention;

FIG. 3 is a perspective view of a cartridge nest and injector including an absorbent material located about a portion of the injector in accordance with one embodiment of the present invention;

FIG. 4 is a partial side sectional view of a secure syringe illustrating a locking mechanism in accordance with one embodiment of the present invention;

FIG. 5 is a partial perspective view of components of a secure syringe illustrating the locking mechanism in an engaged or locked orientation in accordance with one embodiment of the present invention;

FIG. 6 is a partial perspective view sectional view of components of a secure syringe illustrating the locking mechanism in a disengaged or unlocked orientation in accordance with one embodiment of the present invention;

FIG. 7 is an example of a user interface for a mobile device along with a secure syringe in accordance with one embodiment of the present invention;

FIG. 8 is an example of a user interface for a mobile device along with a secure syringe in accordance with one embodiment of the present invention;

FIG. 9 is a flowchart illustrating operations of a method for administering medication through a secure syringe in accordance with one embodiment of the present invention;

FIG. 10 is a flowchart illustrating operations of a method for notifying a user to administer a medicinal treatment in accordance with one embodiment of the present invention;

FIG. 11 is a flowchart illustrating operations of a method for setting and modifying scheduling and dosages of medicinal treatments administered through a secure syringe in accordance with one embodiment of the present invention;

FIG. 12 is a flowchart illustrating operations of a method for adding and editing user security profile data in accordance with one embodiment of the present invention;

FIG. 13 is a flowchart illustrating operations of a method for auditing the administration of medicinal treatments in accordance with one embodiment of the present invention;

FIG. 14 is a flowchart illustrating operations of a method for manufacturer and supply chain authentication in accordance with one embodiment of the present invention;

FIG. 15 is a flow chart illustrating operations of a method for collecting, storing, and transmitting captured temperature information to a database in accordance with one embodiment of the present invention;

FIG. 16 is a flow chart illustrating operations of a method for locating and identifying multiple users within a specified physical proximity of one another in accordance with one embodiment of the present invention;

FIG. 17 is flow chart illustrating operations of a method for uploading medical testing results to a database and alerting individuals of risk of contact with a person with a health ailment in accordance with one embodiment of the present invention;

FIG. 18 is a flow chart illustrating operations of a method for collecting and storing health information and sending real time health information to entities in accordance with one embodiment of the present invention;

FIG. 19 is flow chart illustrating operations of a method for collecting and storing health information and displaying the collected health information to the user in accordance with one embodiment of the present invention; and

FIG. 20 is a block diagram of an example embodiment of a computer system upon which embodiments of the inventive subject matter can execute in accordance with one embodiment of the present invention;

### DESCRIPTION OF THE INVENTION

The invention will now be described with reference to the drawing figures, in which like reference numerals refer to like parts throughout. For purposes of clarity in illustrating the characteristics of the present invention, proportional relationships of the elements have not necessarily been maintained in the drawing figures. It will be appreciated that any dimensions included in the drawing figures are simply provided as examples and dimensions other than those provided therein are also within the scope of the invention.

The following detailed description of the invention references specific embodiments in which the invention can be practiced. The embodiments are intended to describe aspects of the invention in sufficient detail to enable those skilled in the art to practice the invention. Other embodiments can be utilized, and changes can be made without departing from the scope of the present invention. The present invention is defined by the appended claims and the description is, therefore, not to be taken in a limiting sense and shall not limit the scope of equivalents to which such claims are entitled.

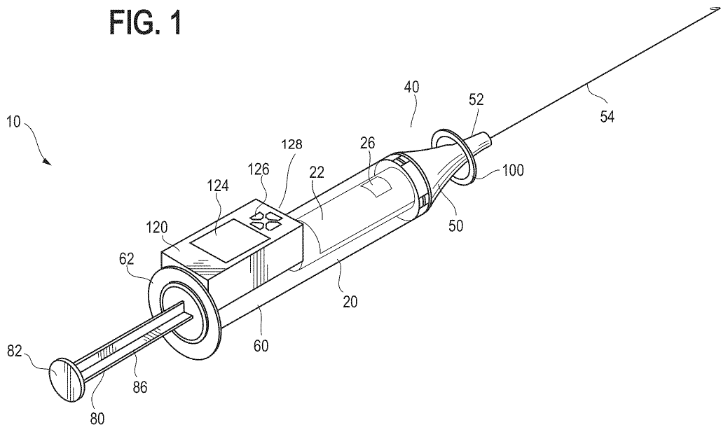

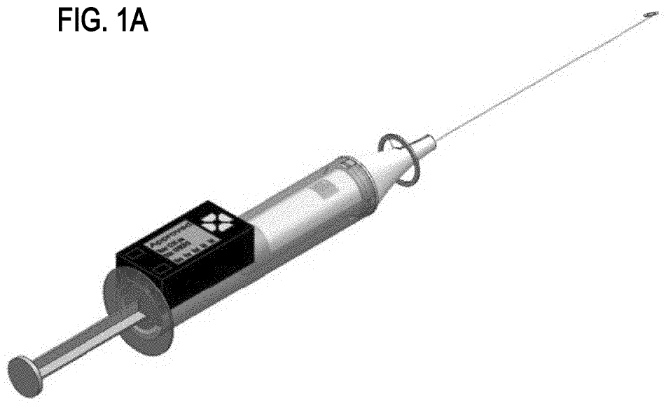

The present invention generally relates to a secure device 10 for administering medications, vaccines, or other active agents to a patient, in a manner that reduces or eliminates abuse or misuse. As used herein, medication or medicine may refer to pain relief medication, vaccines, steroids, or other active agents used in the medical and health fields, which are known or have yet to be discovered. Secure device 10 can further allow for effective and efficient monitoring of the administration of such medications, including the type, dosage, frequency, efficacy, and other metrics. In one embodiment of the present invention, and as demonstrated in FIGS. 1-2, secure device 10 may be comprised of several components and parts that can be assembled together to create a constructed secure device 10. In one embodiment, and as illustrated in the Figures, secure device 10 may comprise a medicine cartridge 20, a cartridge nest 40, an injector 54, a syringe body 60, a plunger actuator 80, a nose guard 100, and an electronics subassembly 120, however, it will be appreciated that other configurations and embodiments are possible.

FIGS. 1-2 demonstrate one embodiment of secure device 10 comprising a medicine cartridge 20. Medicine cartridge 20 may be used as a housing unit for medication that can be administered to a patient, and can be adapted to be used multiple times or alternatively may be adapted to be used a single time. Medicine cartridge 20 may generally consist of a medicine cartridge housing body 22 for the storing of medication until secure device 10 is used to administer the medication. Medicine cartridge housing body 22 may comprise a generally cylindrical cross-sectional shape, however, other geometric shapes and designs are possible and may vary depending on the embodiment. To prevent misuse or abuse of medication, medicine cartridge 20 may further comprise a unique keyed pattern 24 located in the interior of medicine cartridge housing body 22. Unique keyed pattern 24 may comprise a general design to match further components of secure device 10, that when acting together in unison may reduce or eliminate non-authorized or proper administration of medication.

To further prevent or reduce misuse or abuse of medication, medicine cartridge 20 may also contain integrated passive electronics in the form of a barcode or radio frequency identification (“RFID”) chip 26. RFID chip 26 may be located on the exterior surface of medicine cartridge housing body 22, or alternately may be located in the interior of medicine cartridge housing body 22, depending on the embodiment. RFID chip 26 may communicate with a compatible software, application, or other method of electronic communication, whether external or internal to secure device 10, such that secure device 10 may interchange between an active mode for administering medication and a locked mode which prevents the administration of medication to the patient. RFID chip 26 may further be used to store unique data information, digital security codes, or other relevant information in aiding both the administration and monitoring of medication to the patient.

As demonstrated in FIG. 1, medicine cartridge 20 may be housed, stored, or otherwise located in the interior of a housing unit, or cartridge nest 40, which may generally consist of a cartridge nest body 44. Cartridge nest body 44 may be of a cylindrical, triangular, rectangular, or other polygonal or suitable cross-sectional shape, such that the medicine cartridge 20 may be received therein. Cartridge nest 40 may be made of a plastic, rubber, polymer, other suitable materials, or combination thereof. A cartridge nest cover 56 may further be used to removably secure medicine cartridge 20 inside cartridge nest 40, and may further partially or fully cover medicine cartridge 20 while placed inside cartridge nest 40. Cartridge nest cover 56 may be of a suitable shape and design such that the medicine cartridge 20 can be removably placed within the cartridge nest 40. Cartridge nest 40 may further comprise a cartridge nest channel 52, located at the administration end of secure device 10, that may be used for the transfer of medication from medicine cartridge 20 to the patient.

In one embodiment and as illustrated in FIGS. 1-2, secure device 10 may further comprise a seal or top face seal 30, which may provide a sealing effect between cartridge nest 40 and medicine cartridge 20. Top face seal 30 may also provide a pre-load to a top retention shelf 42 inside cartridge nest 40, thereby enabling the medicine cartridge 20 to fit more securely in cartridge nest 40. Top face seal 30 may serve as a backup to prevent medication, fluid, or other liquids from leaking into the rest of cartridge nest 40 and out of cartridge nest cover 56. Also, in reference to FIGS. 1-2, a bottom face seal 58 may be incorporated to help ensure that medication, while administered to the patient, does not leak into cartridge nest body 44 and potentially through cartridge nest cover 56. Additionally, a disposable, perforated seal 90 may help contain the medication in medicine cartridge 20 until ready to be administered.

Cartridge nest 40 may further comprise at least one cartridge nest tab 48 for locking or otherwise attaching or securing cartridge nest 40 to syringe body 60 of secure device 10. In one embodiment of secure device 10, syringe body 60 may slide over cartridge nest 40 until it reaches the cartridge nest tab 48. The cartridge nest tab 48 may be designed in a way such that it creates a mechanical fastening, thereby securing the syringe body to the cartridge nest. However, it will be appreciated that other methods of fastening are within the scope of the present invention and may be used instead.

In one embodiment, cartridge nest 40 may further comprise an application tip 50 to aid in the administration of medication. Application tip 50 may be formed of a soft rubber, plastic, polymer, other suitable flexible material, or combinations thereof. Application tip 50 may be adapted for providing a comfortable method for applying medicine to the patient via a nasal cavity. In one such embodiment, application tip 50 may comprise a generally contoured shape, adapted for entering a nostril of a patient.

Cartridge nest 40 may further comprise an application apparatus for the injection or administration of medication to the patient. In one embodiment of secure device 10, the application apparatus may be an injector 54, which may be a flexible application injector designed to be inserted into the nasal cavity of the patient, such that medicine may be pushed through injector 54 and injected into the nasal cavity of the patient. Injector 54 may extend generally from the application tip 50. Injector 54 may be made of an appropriate length for individual patients, such that it may be long enough to enter the nasal cavity of the patient. Injector 54 may be made from a variety of different materials, and may be made of a plastic polymer, glass, rubber, combinations thereof, or other suitable materials now known or hereafter developed. It will be appreciated that in alternate embodiments of secure device 10, injector 54 may be of different designs, and may instead be a syringe, cannula, or other apparatus for administering medication. By way of non-limiting examples, some vaccines, such as Live Attenuated Influenza or SARS-CoV-2, are administered using a more superficial application tip. In such an embodiment, injector 54 may comprise a design for injection into other locations, such as muscle, veins, arteries, tissue, subcutaneous or dermal layer of skin, or other desired locations.

Secure device 10 may further comprise a syringe body 60, as illustrated in FIGS. 1-2, to further aid in administering medication or active agents to the patient. Syringe body 60 may be cylindrical, triangular, rectangular, or other polygonal or suitable cross-sectional shape in design, depending on the embodiment. As illustrated in FIGS. 1-2, syringe body 60 may generally surround at least a portion of both medicine cartridge 20 and cartridge nest 40, and may aid in the handling of secure device 10. To further aid in handling and use, syringe body 60 can also include a syringe handle 62 for improved fit, comfort, and usability of secure device 10. The design of the syringe handle 62 can vary based on the embodiment and may be designed as having a circular, triangular, rectangular, or other polygonal or suitable shape. Syringe body 60 can be made of a high strength plastic, rubber, polymer, other material, or combinations thereof, and in one embodiment syringe body 60 is made out of a high strength plastic. Syringe body 60 may optionally be reusable and contain electronics for security and data collection systems, or optionally, may be adapted to be disposable and for single use.

In one embodiment of secure device 10, syringe body 60 may further comprise pass-through indentions, recessed areas, or channels to aid in locking and unlocking secure device 10. By way of non-limiting example, syringe body 60 may include at least one syringe body locking channel 64 that permits a retractable feature from an electronics subassembly 120 to pass through in order to prevent a plunger actuator 80 from moving and, thus, preventing plunger actuator 80 from providing the pressure to inject medicine into the patient. In one embodiment of secure device 10, syringe body 60 may further comprise a uniquely shaped passage 66 designed so that it may be compatible with unique keyed pattern 24 of medicine cartridge 20 for providing a security component to secure device 10.

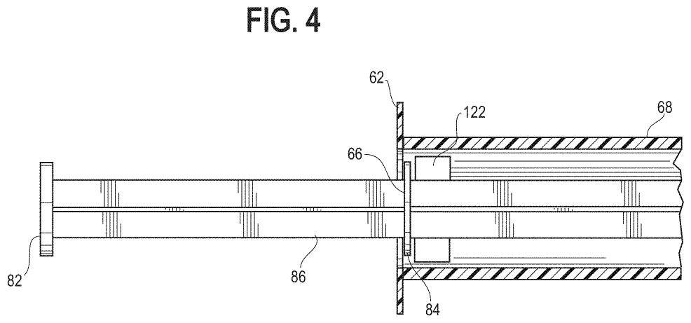

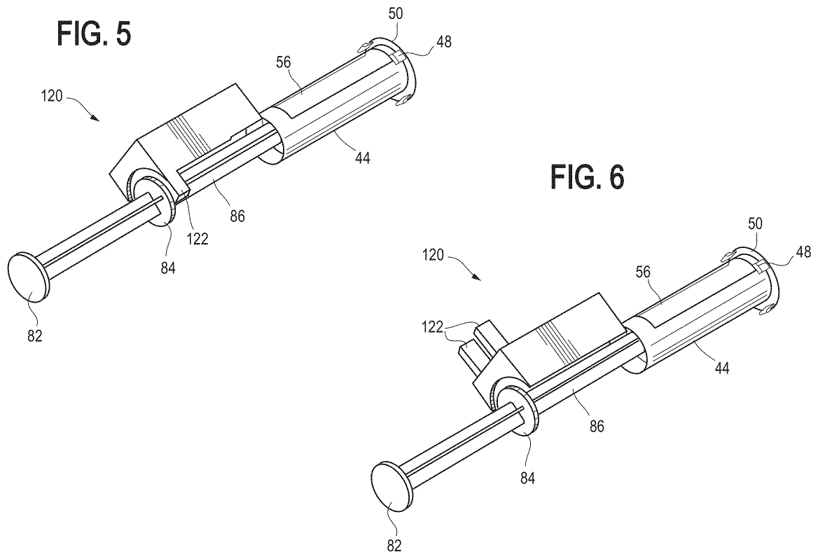

In one embodiment, secure device 10 may further comprise a plunger actuator 80 that can be used to administer medication from medicine cartridge 20 to the patient. Plunger actuator 80 can be uniquely keyed, coded, shaped, or otherwise uniquely identified, such that it is only compatible with a unique or particularized medicine cartridge 20 and/or syringe body 60. Specifically, plunger actuator 80 may be of a compatible design as uniquely keyed pattern 24 and uniquely shaped passage 66. The design of one embodiment of plunger actuator 80 is illustrated in FIGS. 1-6, and may comprise a generally cross-shaped design. However, it will be appreciated that other shapes and designs are possible, depending on the embodiment. Plunger actuator 80 may be disposable and may be disposed of after one use, or alternatively it may be reusable.

In one embodiment, plunger actuator 80 may be retained by syringe body 60, such that it is permanently positioned or placed within syringe body 60. In another embodiment, plunger actuator 80 may be removable from syringe body 60 and may be positioned only when secure device 10 is ready to be in use. It will be appreciated that secure device 10 could utilize other methods of injection, and that plunger actuator 80 is not intended to be a limiting design of the present invention.

Plunger actuator 80 may be formed of plastic, rubber, polymer, other suitable materials, or combination thereof. Plunger actuator 80 may comprise a finger engagement surface 82 at the distal end thereof, to aid the operator in handling secure device 10. Plunger actuator 80 may further comprise a plunger tab 84, located at a point on an elongated plunger shaft 86 to help ensure plunger actuator 80 stays retained within the syringe body 60. In one embodiment of the present invention, plunger shaft 86 is of a cross-sectional shape matching the cross-sectional shape of the specially-shaped channel 66 of the syringe body 60 and the medicine cartridge pattern 24. However, it will be appreciated that the design of plunger shaft 86 could be of a matching shape, size, and design, such that it is compatible with unique keyed patter 24 and uniquely shaped passage 66, to allow only approved plunger actuators 80 to work with secure device 10. At a second end, opposite from plunger tab 84, plunger actuator 80 may further comprise a plunger seal 88, for pressurizing medicine located in medicine cartridge 20. Plunger actuator 80 may act as the top pressure seal in medicine cartridge 20 to inject medicine or fluid into a patient. When plunger shaft 86 feeds through medicine cartridge 20, plunger seal 88 can pressurize the liquid medicine within the medicine cartridge 20 and push the medicine out.

As illustrated in FIGS. 1-2, secure device 10 may optionally and additionally comprise a nose guard 100 in an embodiment of secure device 10 used to administer medication in the nasal passage. In such an embodiment, nose guard 100 may prevent or reduce the chance of secure device 10 being inserted too far into the patient's nasal cavity. Nose guard 100 may be comprised of a nose guard ring 102 and a nose guard stem 104. Nose guard ring 102 may be designed to be generally circular in design, or it may be triangular, rectangular, or other polygonal or suitable shapes, such that the nose guard 100 may be used to prevent application tip 54 from penetrating too deeply into the nasal passage. Nose guard stem 104 may extend from nose guard ring 102 towards the interior of nose guard 100. Nose guard stem 104 may be used to secure nose guard 100 to secure device 10. In one embodiment of the invention, nose guard 100 may be connected to application tip 50, by inserting the nose guard stem 104 into a grooved channel and sliding nose guard stem 104 through the grooved channel until the nose guard stem 104 locks in place, thereby securing nose guard 100 to secure device 10. Nose guard 100 may be a separate piece that can be affixed to secure device 10, or alternately it may be a separate piece assembled to the disposable cartridge nest 40 in order to prevent the application tip 50 and applicator 54 from inserting too deeply into the nasal cavity of the patient.

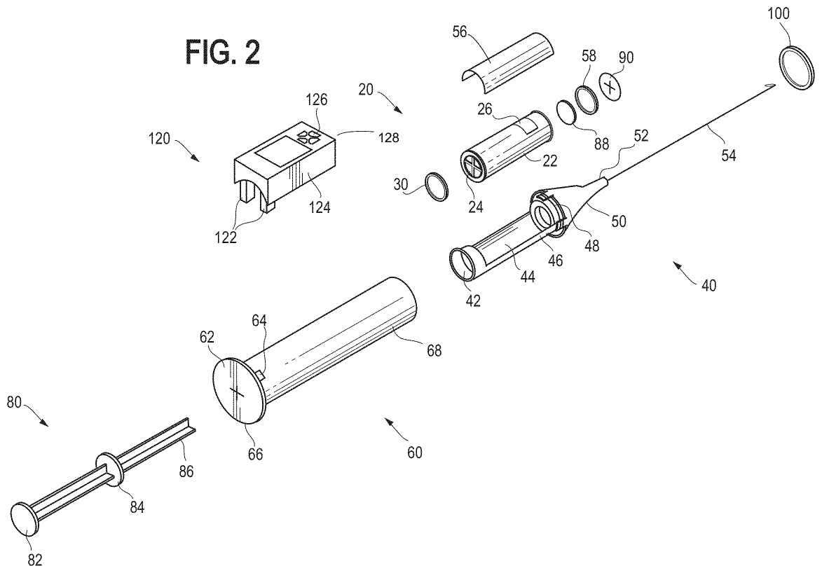

Secure device 10 may optionally and additionally comprise a means for collecting a biological fluid specimen from the patient. As such, the secure device 10 can include a biological fluid specimen collector, such as a nasal fluid specimen collector, for example. In one embodiment, as illustrated in FIG. 3, secure device 10 can include an absorbent material, which may be in the form of a swab 53 located, embedded, or otherwise attached to the surface of injector 54. The swab may be a sterile cotton swab or the like and can be located near the cartridge nest channel 52 (i.e., at a proximal end of the injector 54) to facilitate the collection of biological fluid specimen for nasopharyngeal and/or oropharyngeal sampling. After the medication has been dispensed into the nose cavity, the cartridge nest 40, injector 54 and/or swab 53 containing the collected nasal specimen is adapted for removal, and may be removed, from a remainder of the device 10 and placed in a secured sterile sampling tube to be sent for further laboratory testing of the specimen.

In one embodiment, secure device 10 may further comprise an integrated electronics subassembly 120 that may house security and data acquisition electronics and other components. Electronics subassembly 120 may be attached to syringe body 60 through a fastening method including an adhesive or it may be attached through a mechanical means, among other methods. In exemplary embodiments, electronics subassembly 120 may be built or manufactured or otherwise permanently affixed to the syringe body 60. As demonstrated in FIGS. 1-2, electronics subassembly 120 may be located at the distal end of syringe body 60, near syringe handle 62 or otherwise opposite from the administration end of secure device 10.

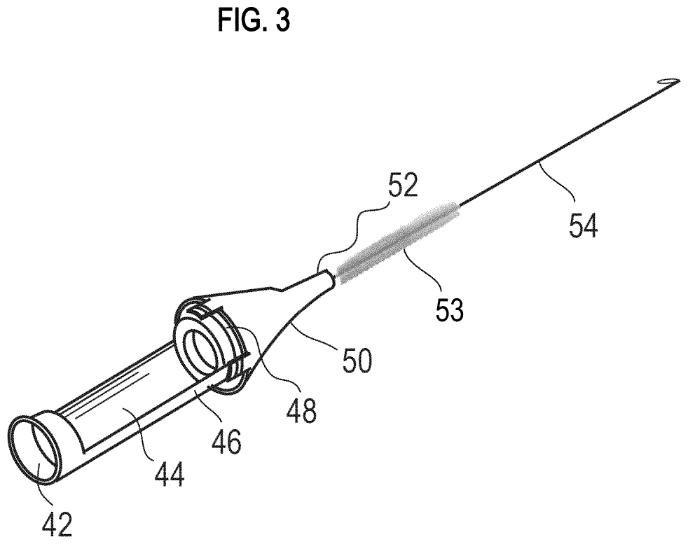

As demonstrated in FIGS. 4-6, electronics subassembly 120 may comprise one or more high strength levers 122 for locking secure device 10 and preventing plunger actuator 80 from pushing through secure device 10 and medicine cartridge 20, thereby preventing the injecting of medicine into the patient. To achieve this effect, electronics subassembly 120 may selectively stroke or otherwise extend into syringe body 60 and prevent the retaining surface on plunger actuator 80 from moving axially and pressurizing medicine cartridge 20. High strength levers 122 may be retractable such that when medicine is to be administered in accordance with scheduling and security procedures, high strength levers 122 may no longer retain plunger actuator 80 so that medicine can be administered. In the illustrated embodiment, high strength levers 122 may enter syringe body 60 through syringe body channel 64 and act as stopping point or block, preventing plunger actuator 80 from pushing forward. When medicine is ready to be administered, high strength levers 122 may be retracted through syringe body channel 64, allowing plunger actuator 80 to extend through syringe body 60 and medicine cartridge 20 to administer the medicine.

Electronics subassembly 120 may further incorporate a display screen 124 to show status and menus and may be further used to control the locking mechanism and other functionalities of secure device 10. Electronics subassembly 120 may further comprise direction and selection buttons 126, allowing the user to navigate through menus of electronics subassembly 120 and select different options. Direction and selection buttons 126 may be comprised of directional buttons, such as up, down, and side-to-side buttons, or it may incorporate a wheel, touch screen, or other directional interfaces. In one embodiment, directional selection button 126 comprise four buttons, corresponding to an up, down, left, and right selection.

It will be appreciated that integrated electronics subassembly 120 is optimally designed to incorporate additional and further electronic or medical components. Electronics subassembly 120 may also be adapted for interfacing with a peripheral sensor, such as that included in a wearable, capable to detect and transmit the patient vital signs information data. One optional electrical component is an electronic device for the measuring a patient's temperature, prior to, during, and/or after administration of medication. By way of a nonlimiting example, integrated electronics subassembly 120 may comprise an infrared proximity temperature (“IRPT”) sensor 128 for taking and recording the patient's temperature, before, during, and/or after dosage administration. It will be appreciated that IRPT sensor 128 may be located near or at the administration end of integrated electronics subassembly 120, such that a passive infrared device within the IRPT sensor assembly 128 can be directed to and ultimately interact with the IR radiation from the forehead or other body part of the patient for measuring and recording the patient's temperature. It will be appreciated that the temperature reading of the patient captured by IRPT sensor 128 could be directed to display screen 124, allowing the operator to read the patient's temperature. However, it will be further appreciated that the recorded temperature, vital signs, other information, and/or date could be stored on a memory device located in integrated electronics subassembly 120, and further yet, the recorded temperature could be transmitted to a cloud, web-based, or otherwise remote location for storage and recordation. Secure device 10 may further implement, comprise, or consist of other electronic, or alternatively non-electronic, tools and medical apparatus for any of assisting in administering the medication, collecting health metrics and vitals, storing health information, and/or disseminating or transmitting health information to a storage database or to electronics subassembly 120. In alternate embodiments, electronics subassembly 10 could comprise additional medical or electronic components to either assist in administering medicine or for collecting and storing other health information.

Electronics subassembly 120 can be further adapted for communicating with RFID chip 26 integrated in medicine cartridge 20. Electronics subassembly 120 may have the ability to identify a designated medicine cartridge 20 and permit the syringe actuation mechanism only when desired by the operator and approved by a medical professional or proper administrator. Secure device 10 may be designed and implemented to prevent distribution or application of medication from medicine cartridge 20 without the appropriate identification of patient and/or administrator, medicine, dose, schedule, among other pre-approved parameters. Medicine cartridge 20 may provide information, such as a unique code, barcode, identification number, or other method of identification as part of its unique identification. Electronics subassembly 120 may then read and verify the information of medicine cartridge 20 information and, based on the information received, may unlock secure device 10 to administer medicine.

In one embodiment of the present invention, secure device 10 may be collapsible, disassembled, or otherwise shortened in order to reduce its overall length for storage and transportation. For example, application tip 50 can become a telescopic device and be stored within the tip of cartridge nest 40. Plunger actuator 80 can also become a telescopic device, and the length of plunger actuator 80 beyond the retention surface can collapse into the length that is within syringe body 60.

When fully assembled, secure device 10 may be used to inject or otherwise administer medicine into a patient securely, safely, and in a manner that limits or prevents abuse. In one embodiment, as illustrated in FIGS. 1-2, secure device 10 may initially start in a secured and locked state. In this state, the electronics subassembly 120 may function as an interference locking mechanism in which high strength levers 122 extend into syringe body 60, locking in place in syringe body channel 64. High strength levers 122 in this position may create an interference in syringe body 60 and may prevent plunger actuator 80 from pushing through both syringe body 60 and medicine cartridge 20, thereby preventing the medicine from being pushed and transferred through the cartridge nest channel 52 and injector 54. When the patient receives authorization from a medical professional or administrator, or when a medical professional or administrator unlocks secure device 10 using electronics subassembly of medicine cartridge 20, the patient, medical professional, or administrator may use the direction and selection buttons 126 to unlock secure device 10 and retract high strength levers 122 from syringe body channel 64. When high strength levers 122 are removed from syringe body channel 64, plunger actuator 80 may then be pushed through secure device 10 to administer medicine.

After unlocking secure device 10, it may then be used to administer medicine to a patient through injector 54. In one embodiment, secure device 10 may be used to administer medicine into the patient's nasal cavity. In such an embodiment, injector 54 may be of an appropriate length, depending on the patient, to enter the nasal cavity of the patient. Injector 54 may be inserted into the nasal cavity of the patient until application tip 50 reaches the nostril of the patient. Because of its contoured design, application tip 50 may enter safely and comfortably into the nostril of the patient. To prevent injector 54 from entering too far into the patient, nose ring 100 may act as a termination point when coming in contact with the nostril. When secure device 10 and/or injector 54 is in the appropriate position in the nasal cavity, plunger actuator 80 may then be used to administer medicine. The patient, medical professional, or administrator may use plunger actuator finger engagement surface 82 and syringe body handle 62 to press plunger actuator 80 through the device. Plunger seal 88 may be used to push the medicine from medicine cartridge 20 through cartridge nest channel 52. When pushed through cartridge nest channel 52, the pressurized medicine or fluid may break the disposable, perforated seal 90. The medicine may continue to be pushed through the cartridge nest channel 52 through injector 54 and into the nasal cavity of the patient. After the medicine is administered, secure device 10 may be removed from the patient. It will be appreciated the other embodiments and methods of use are within the scope of the present invention, and the present invention may be used to administer medicine to other locations in a patient, including, but not limited to the dermal layer, muscle, veins, and arteries, among other parts of the patient.

Secure device 10 may be configured so that electronics subassembly 120 may communicate with a dedicated software application installed on a smartphone, tablet, or other mobile device (not shown). The electronics subassembly 120 may also communicate with one or more dedicated sensor-enabled peripheral devices (not shown) capable of detecting a patient's vital signs such as body temperature, pulse heart rate, blood pressure, blood oxygen saturation, blood glucose level, and other vital signs. However, it will be appreciated that the application and/or its user interface may also be installed or displayed on any suitable device, including but not limited to, a laptop computer, desktop computer, wearable device, smart appliance, infotainment system, gaming system, television, media player, e-reader, or any other electronic device suitable for running software applications. Electronics subassembly 120 may have the ability to be paired with and communicate via Bluetooth connectivity, Wi-Fi connectivity, broadband cellular networks and/or other wireless or hardwired communication standard with a device, such as a smartphone or tablet device.

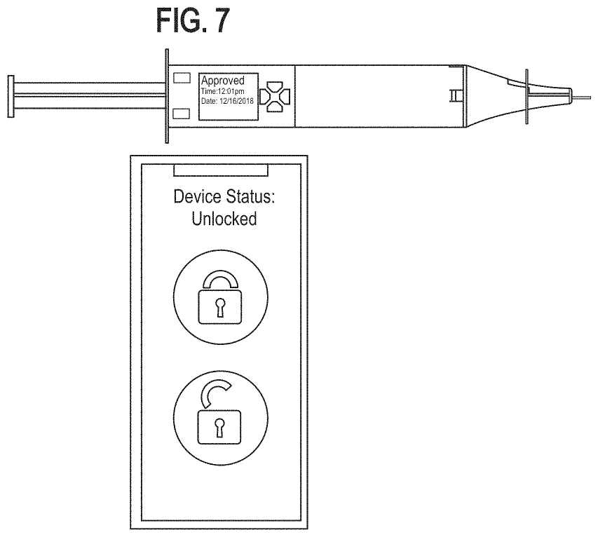

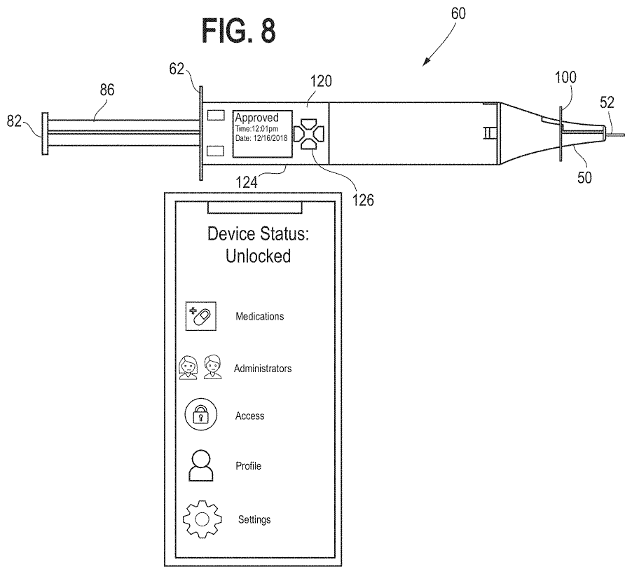

The dedicated software application developed for the smartphone, tablet, or other devices can have the ability to interact with the patient and/or the administrator. The dedicated software application provides a convenient user interface allowing pre-programming and eventual use of secure device 10. Thus, the patient information, medication, dosage schedule and other parameters can be pre-programmed and downloaded to electronics subassembly 120. As a result, secure device 10 can be used independently by the patient and/or administrator, as long as the patient is compliant with the pre-approved settings. In addition, the dedicated software application can utilize built in camera and/or microphone components of a smartphone, tablet, or other device as a photographic and/or speech recognition security feature. The authorized patient or administrator can be permitted to set a unique customizable user profile in the dedicated software application with the ability to store the user's facial pictures, speech recordings, fingerprint data, retina data, vein or other eye data, or other suitable biometric information of the user. These pictures and/or voice recordings may be utilized to perform a secure user authentication by which to permit usage of the secure device 10. As a result, in one embodiment, the software application will only permit activation of secure device when: (1) the designated user is identified; (2) the correct medication cartridge is inserted in the syringe; (3) the pre-programmed dosage is prescribed; and (4) the pre-programmed scheduled dosage time period is active. The dedicated software application can also securely transmit the medication information, vital signs information and patient usage information to a medical provider's electronic medical records. As a result, the patient's medical record can be updated in real-time with the medication usage information. Examples of different methods of use of secure device 10 and the dedicated software application are illustrated in FIGS. 7-8.

For each medicine cartridge 20, a manufacturer may populate the electronic section of the medicine cartridge 20 with a unique serial and hash data identifiers which enable the medicine cartridge 20 to be registered as a digital asset on a Blockchain network. Once each medicine cartridge is registered to the Blockchain by the manufacturer, its ownership can be readily transferred and tracked in the distribution supply chain. Such tracking can help to ensure that each medicine cartridge 20 arrives at its intended location, thus increasing the chance of proper use.

It will be appreciated that in addition to providing a secure method of the administering medicine, secure device 10 may further be used to collect, store, and transmit health metrics and other data that may be used by patients, health professionals and organizations, and the general population. Secure device 10 may also connect to mobile applications, web-based applications, Blockchain networks, or other networks or software using Wi-Fi, Bluetooth, broadband cellular networks, or other communication means to transmit health metrics and other data in real-time during use of secure device 10. Through its connection and communication to such networks, applications, and/or software, secure device 10 may be used to provide generally real-time data and metrics to health professionals, patients, entities, and others. In one embodiment, secure device 10 may be in electronic communication to the same dedicated software application as described above; however, it will be appreciated that secure device 10 may optionally or additionally connect to a separate software application, web-based service, or other software.

By way of a non-limiting example, in one embodiment secure device 10 may collect health information including without limitation, health metrics including vitals, temperature, or other health information, through IRPT sensor 128 or other methods. Additionally, secure device 10 may collect the dosage, frequency, and other information of the administered medication in medicine cartridge 20. Secure device 10, through electronics subassembly 120, may then transmit the collected information through electronic communication to the dedicated software application. Such information may be accessible to the patient or user through the dedicated software application through use of an electronic device such as a cellular phone, tablet, computer, or other device or alternatively may be displayed on display screen 124 for immediate reading by the patient. Further, in addition to relaying the collected health information back to the patient, the dedicated software application may be used by doctors, health professionals, or others to review such information and/or transmit additional information back to the patient or user. Such additional information may be transmitted to the user via the dedicated software application, in the form of push notifications, alerts, prompts, or other communication methods. Such information transmitted to the user may include, but not limited to, testing results, location of testing facilities, location of transmittable disease outbreaks, and other information. Additionally, the collected health metrics, vitals, and other information recorded or captured by secure device 10, such as a temperature spike, or decreased oxygen levels, may alert the patient that immediate medical attention may be required.

The dedicated software application and secure device 10 may further be used with multiple users, and can further be used in aggregate data collection, as well as providing real-time and up-to-date information to multiple users in a variety of scenarios. To help keep collected health information and other data categorized and associated with a specific user, each individual user may have his or her own Unique Identifier (“UID”) incorporated into the dedicated software application and/or secure device 10 for providing an identifier to a specific and individual user. When health metrics, test results, or other information is uploaded to the dedicated software application, a UID may be attached, connected, or otherwise used to connect such information to a specific user, allowing accurate storage of the information for users, health professionals, and others. UIDs may further act as a barrier between the user and the user's personal data and privacy, to aid in preventing unauthorized disclosures of personal data, health data, or other sensitive information associated with an individual user.

The dedicated software application may be used to monitor users' UID and develop a physical proximity network, developed through active or intermittent scanning of devices connected to the dedicated software application. UIDs may be discoverable through Wi-Fi, Bluetooth, broadband cellular networks, or other methods. The physical proximity network may be used to create an ever-evolving database of the number, location, and distance between multiple users within a specified geographic area. Such information may be collected and stored using the dedicated software application and/or the secure device 10. The physical proximity network may be used to collect data including, but not limited to, tracing the location and spread of health ailments, the distribution of medication and vaccines, recovery rates from ailments, percentages of a population that have been vaccinated, among other things. The physical proximity network, is particularly effective for retroactive and immediate contact tracing. For example, if a user has been identified as having a specific health ailment, particularly an ailment that is transmittable to others, the dedicated software application can send notifications to other users that have been in proximity to the potentially contagious user. The dedicated software application may further provide these at-risk users with medical information such as testing centers, hospital locations, or locations for treatment.

The collected health information may further be transmitted or otherwise accessible to medical professionals, government officials or agencies, or other entities for monitoring and other uses. This collected information may be particularly beneficial for tracking and monitoring transmittable diseases, but it will be appreciated that other uses would also be beneficial. For example, the collected health information may alert such entities to information including, but not limited to: (1) the number of individuals in a specific geographic location suffering from a specific health ailment; (2) the rate of spread of a specific health ailment by individual person; (3) the rate of spread of a specific health ailment by geographic location; and/or (4) the efficacy of administered vaccines or medication. Using secure device 10 and/or the dedicated software application, such information can be provided to medical professionals, government officials or agencies, or other entities in real-time. With real-time information, such entities may formulate strategies for the allocation of medications or vaccines, food, water, or other supplies, as well as monitoring trends of spread, recovery, restrictions, and other relevant information.

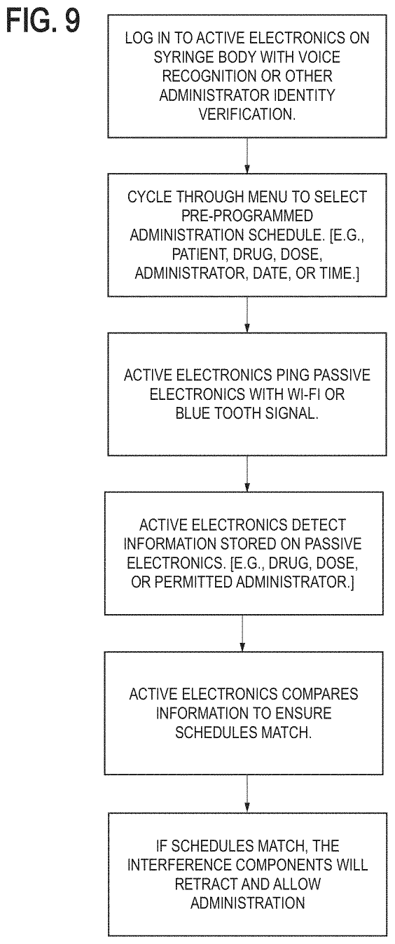

FIGS. 9-14 provide flowcharts of methods for administering, scheduling, notifying, setting up user profiles, auditing, and authenticating. These flowcharts demonstrate the security electronics logic and outline the processes the software and electronics will follow in different circumstances.

One such method is illustrated in FIG. 9. In this method of use, an administrator or medical professional may log in to the operating system or software compatible with the device 10 to activate the integrated electronics on the syringe body 60 with voice recognition or other administrator identity verification. The administrator or authorized user may then cycle through the menu to select pre-programmed administration schedule, with information including without limitation patient information, drug information, dose information, administrator information, date, and time. The active electronics of the device 10 may ping passive electronics with Wi-Fi, Bluetooth, or other methods of signal communication. The active electronics of the device 10 may detect information stored on the passive electronics, including without limitation, patient information, drug information, dose information, administrator information, date, and time. The active electronics can then compare information to ensure that schedules of the active electronics match the pre-programmed administration schedule. If the schedules match, the interference components (i.e., the levers 122) may retract and allow administration of medicine to the patient.

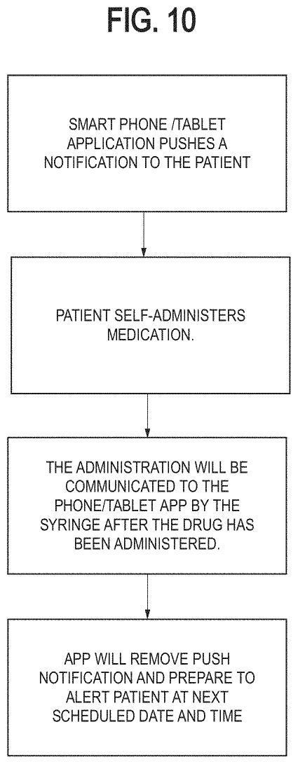

Another such method is illustrated in FIG. 10. In this method of use, a smart phone, computer, tablet or other device application may push a notification to the patient. The patient may then self-administer the medication using the device 10. The administration, medical professional, or other authorization authority may be communicated to the phone, tablet, or other device app by the device 10 after the drug has been administered. After administration, the application may remove the push notification and can then prepare to alert the patient at the next scheduled date and time.

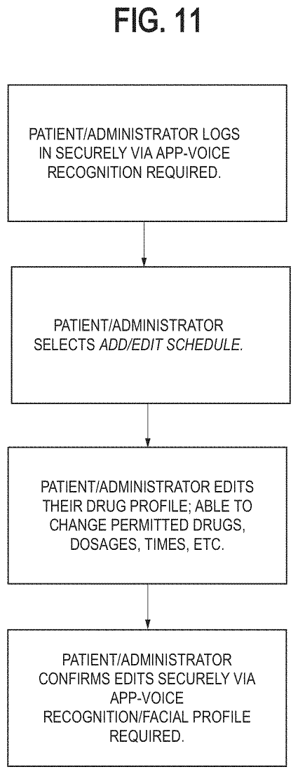

Another such method is illustrated in FIG. 11. In this method of use, the patient or administrator may log in securely to the program using a phone, tablet, or other device application. In this particular method, the application may use voice recognition as a method of security. The patient or administrator may then select a pre-programmed option including without limitation an Add Schedule or Edit Schedule option. The patient or administrator may then edit the drug profile, may change the permitted drugs, the dosages, the times and other options. The patient or administrator may then confirm their option selection or edits by using the application, and for added security this method may use voice recognition or facial recognition.

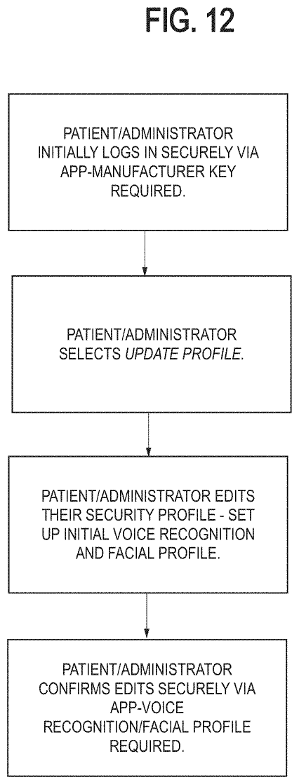

Another such method of use is illustrated in FIG. 12. In this method of use, the patient or administrator may initially log in to the mobile, phone, tablet, or other device application using a manufacturer key. The patient or administrator may then select the Update Profile option on the application. The patient or administrator may then edit their security profile and may further set up initial voice recognition and facial profile. The patient or administrator may then confirm the edits securely via the application, and in this particular method, the patient or administrator may use voice recognition or facial recognition to confirm.

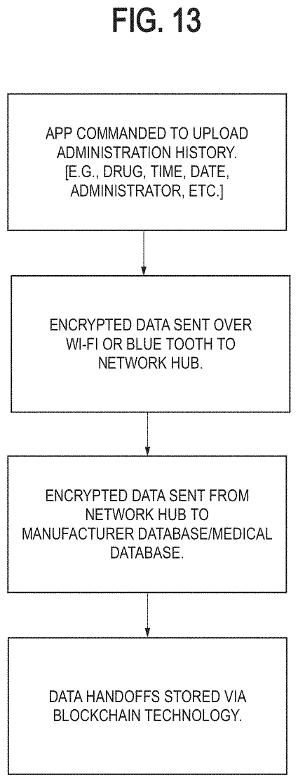

Another such method of use is illustrated in FIG. 13. In this method the patient or administrator may use the application to command an upload of administration history. Such information in the history may include, without limitation, patient information, drug information, dose information, administrator information, date, and time. The information uploaded may be encrypted and the data may be sent over Wi-Fi or Bluetooth to a network hub that may securely store the information. The encrypted data may be sent from the network hub to a manufacturer database or medical database. The handoff of data may be stored via Blockchain technology.

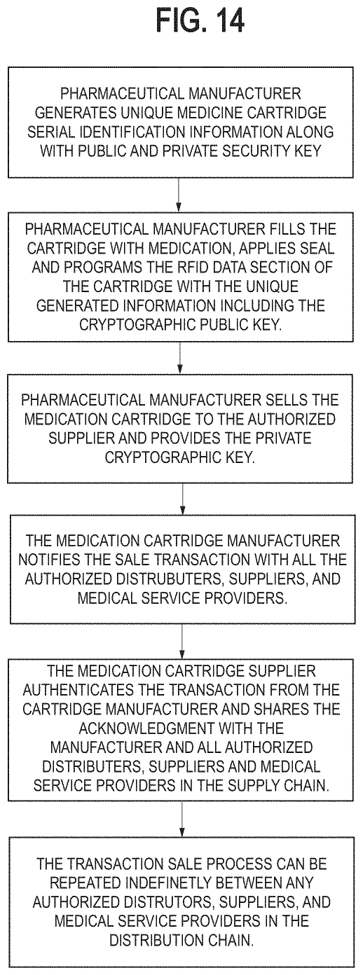

Another such method of use is illustrated in FIG. 14. In this method the pharmaceutical manufacturer may generate the unique medicine cartridge serial identification information along with a public and private security key. The pharmaceutical manufacturer may then fill the medicine cartridge 20 with medication, and apply a seal to the medicine cartridge, and may further program the RFID data section of the medicine cartridge with the unique generated information including the cryptographic public key. The pharmaceutical manufacturer may then sell the medicine cartridge to an authorized supplier and may provide the private cryptographic key. The medicine cartridge manufacturer may then notify all the authorized distributor, supplier, and medical service providers of the sale transaction. The medication cartridge supplier may then authenticate the transaction from the cartridge manufacturer and share the acknowledgment with the manufacturer and all authorized distributors, suppliers, and medical service providers in the supply chain. The transaction sale process can be repeated indefinitely between any authorized distributors, suppliers and/or medical service providers in the distribution chain.

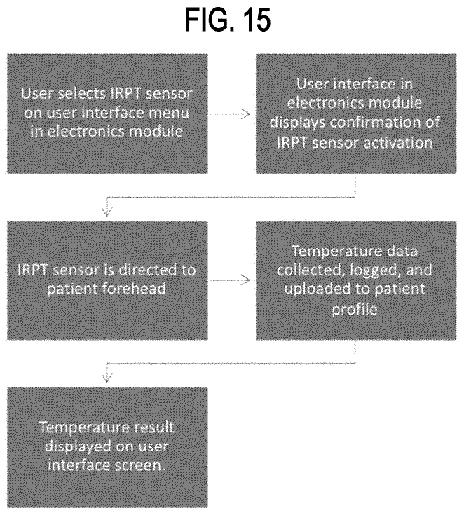

FIGS. 15-19 provide additional flowcharts of methods for administering, scheduling, notifying, auditing, tracing, or otherwise storing health information, metrics, dosages, frequencies, or other information when using secure device 10.

One such method of use is illustrated in FIG. 15. In this method of use, the user or administrator may select an option on electronics subassembly 120 to activate IRPT sensor 128 using the interface method on display screen 124. Display screen 124 may then display confirmation of the activation of IRPT sensor 128, alerting the user or administrator that IRPT sensor 128 is activated and ready for use. The user or administrator may then direct IRPT sensor 128 to the patient's forehead or other applicable body part to determine the body temperature of the patient. The patient's body temperature data may then be collected, logged, or otherwise stored or cataloged. In one such embodiment, the temperature may be uploaded to the patient's medical profile on the dedicated software application or other database. Additionally, the recorded temperature may further be displayed on display screen 124 for immediate viewing by the user, administrator, or patient.

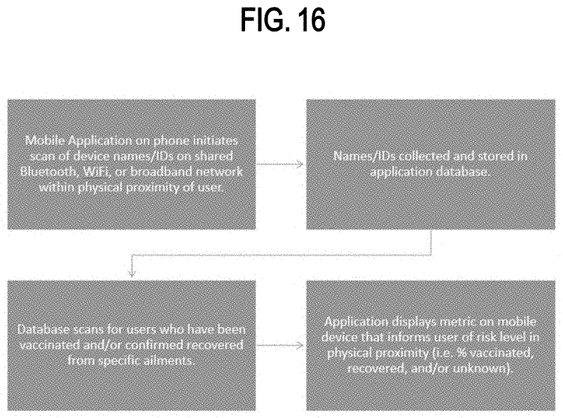

Another such method of use is illustrated in FIG. 16. In this method of use, the patient or user may use the dedicated software application on their cellular phone or other electronic device to initiate a scan of a specified area for discovering one or more other users' UID with electronic devices with the dedicated software application on a shared Bluetooth, Wi-Fi, or broadband network within physical proximity of the initial user. In one embodiment, users of the dedicated software application may have opted-in or otherwise allowed information stored on their cellular device, tablet, or mobile application to be discoverable and shared with other users of the dedicated software application. After initial discovery and identification of users within the specified physical proximity, names, identification numbers, UIDs, or other identifying information is collected and stored in a database for cataloging the location and proximity of users. Additional user information may also be collected and stored, such as administered medications, including vaccines, and current health status of individual users (i.e., whether an individual user is healthy, suffering from a specific ailment, whether the user has recovered from a specific ailment, among other health information). The dedicated software application may then display health and other medical metrics on the user's electronic device, that can inform the user of a determined risk level in the user's physical proximity, resulting from a determination of a number of factors, including without limitation, the percentage of vaccinated users, the number of recovered persons, and other relevant factors.

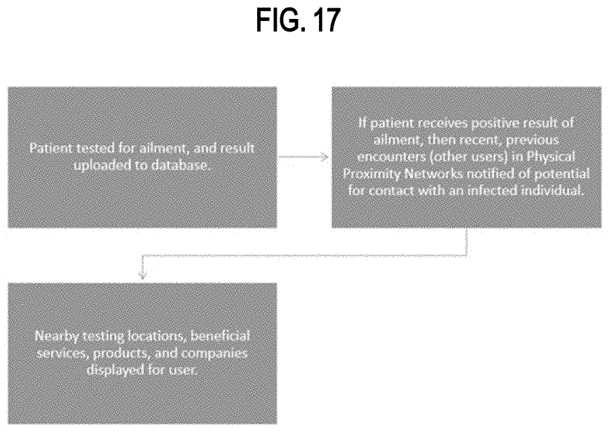

Another such method of use is illustrated in FIG. 17. In this method of use, a patient or user may be tested for a specified health ailment, including without limitation, SARS-CoV-2, HIV, or other viruses or ailments. After testing and receiving the testing results, the patient or user may then upload the results of the test and/or health status to a database using the dedicated software application. If a positive test result of the tested ailment is uploaded, the dedicated software application may provide notification to other users that were recently within the physical proximity network to the tested user, notifying the other users of potential contact with a user with a specified health ailment and alerting the other users that they may be at-risk. The dedicated software application may then provide at-risk users information, including but not limited to nearby testing locations, beneficial services, products, and companies, and other such information.

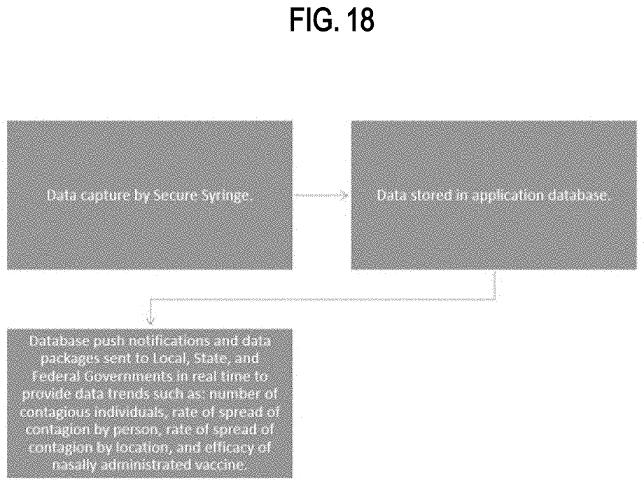

A further method of use is illustrated in FIG. 18. In this method of use, secure device 10 may be used to capture health metrics, vitals, or other information including without limitation temperature, heart rate, blood oxygen level, or other information using IRPT sensor 128 or other apparatus using secure device 10. The captured health information may then be uploaded and stored to a database. The captured health information may be uploaded to the database either directly from secure device 10 or through the dedicated software application. The database may then transmit the health information in the form of push notifications, or other delivery methods, to local state, and federal governments, or other entities or individuals. The dissemination of health information can provide such entities with up-to-date data trends, including but not limited to the number of contagious individuals, rate of spread of contagion by person, rate of spread of contagion by location, and efficacy of administered medications. The electronics subassembly 120 can also be adapted for notifying at least one of a patient, healthcare provider and administrator when at least one vital sign parameter meets a predetermined threshold.

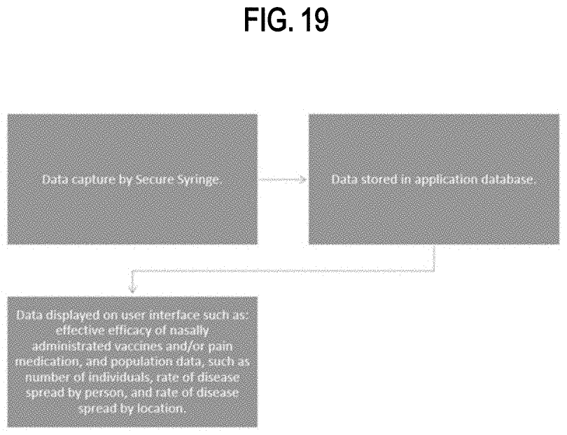

Another such method of use is illustrated in FIG. 19. In this method of use, secure device 10 may be used to capture health metrics, vitals, or other information including without limitation temperature, heart rate, blood oxygen level, or other information using IRPT sensor 128 or other apparatus. The captured health information may then be uploaded and stored to a database. The captured health information may be uploaded either directly from secure device 10 or through the dedicated software application. The captured health information may also be displayed on display screen 124 alerting the user, patient, and/or administrator with information including, but not limited to, the efficacy of the administered medication and population data such as rate of disease spread by person and rate of disease spread by location.

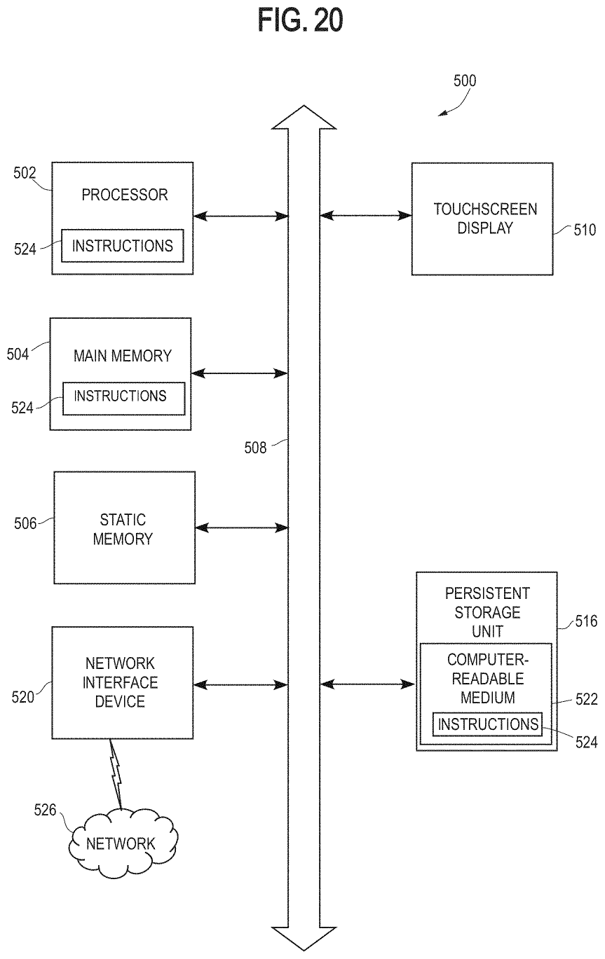

With reference to FIG. 20, an example embodiment of a method of using the present invention extends to a machine in the example form of a computer system 500 within which instructions for causing the machine to perform any one or more of the methodologies discussed herein may be executed. In alternative example embodiments, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment. Further, while only a single machine is illustrated, the term “machine” shall also be taken to include any collection of machines that individually or jointly execute a set (or multiple sets) of instructions to perform any one or more of the methodologies discussed herein.

The example computer system 500 may include a processor 502 (e.g., a central processing unit (CPU), a graphics processing unit (GPU) or both), a main memory 504 and a static memory 506, which communicate with each other via a bus 508. The computer system 500 may further include a touchscreen display unit 510. In example embodiments, the computer system 500 also includes a network interface device 520.

The persistent storage unit 516 includes a machine-readable medium 522 on which is stored one or more sets of instructions 524 and data structures (e.g., software instructions) embodying or used by any one or more of the methodologies or functions described herein. The instructions 524 may also reside, completely or at least partially, within the main memory 504 or within the processor 502 during execution thereof by the computer system 500, the main memory 504 and the processor 502 also constituting machine-readable media.

While the machine-readable medium 522 is shown in an example embodiment to be a single medium, the term “machine-readable medium” may include a single medium or multiple media (e.g., a centralized or distributed database, or associated caches and servers) that store the one or more instructions. The term “machine-readable medium” shall also be taken to include any tangible medium that is capable of storing, encoding, or carrying instructions for execution by the machine and that cause the machine to perform any one or more of the methodologies of embodiments of the present invention, or that is capable of storing, encoding, or carrying data structures used by or associated with such instructions. The term “machine-readable storage medium” shall accordingly be taken to include, but not be limited to, solid-state memories and optical and magnetic media that can store information in a non-transitory manner, i.e., media that is able to store information. Specific examples of machine-readable storage media include non-volatile memory, including by way of example semiconductor memory devices (e.g., Erasable Programmable Read-Only Memory (EPROM), Electrically Erasable Programmable Read-Only Memory (EEPROM), and flash memory devices); magnetic disks such as internal hard disks and removable disks; magneto-optical disks; and CD-ROM and DVD-ROM disks. A machine-readable storage medium does not include signals.

The instructions 524 may further be transmitted or received over a communications network 526 using a signal transmission medium via the network interface device 520 and utilizing any one of a number of well-known transfer protocols (e.g., FTP, HTTP). Examples of communication networks include a local area network (LAN), a wide area network (WAN), the Internet, mobile telephone networks, Plain Old Telephone (POTS) networks, and wireless data networks (e.g., Wi-Fi and WiMax networks). The term “machine-readable signal medium” shall be taken to include any transitory intangible medium that is capable of storing, encoding, or carrying instructions for execution by the machine, and includes digital or analog communications signals or any other intangible medium to facilitate communication of such software.

From the foregoing, it will be seen that this invention is one well adapted to attain all the ends and objects hereinabove set forth together with other advantages which are inherent to the structure and method. It will be understood that certain features and sub combinations are of utility and may be employed without reference to other features and sub combinations. This is contemplated by and is within the scope of the claims. Since many possible embodiments of the invention may be made without departing from the scope thereof, it is also to be understood that all matters herein set forth or shown in the accompanying drawings are to be interpreted as illustrative and not limiting.

The constructions described above and illustrated in the drawings are presented by way of example only and are not intended to limit the concepts and principles of the present invention. Thus, there has been shown and described several embodiments of a novel invention. As is evident from the foregoing description, certain aspects of the present invention are not limited by the particular details of the examples illustrated herein, and it is therefore contemplated that other modifications and applications, or equivalents thereof, will occur to those skilled in the art. The terms “having” and “including” and similar terms as used in the foregoing specification are used in the sense of “optional” or “may include” and not as “required”. Many changes, modifications, variations and other uses and applications of the present construction will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

What is claimed is:

1. A secure device for delivering medications, the device comprising:
a syringe body;
a cartridge nest;
a plunger actuator; and
a biological fluid specimen collector.

2. The device of claim 1, wherein the biological fluid specimen collector is a nasal fluid specimen collector.

3. The device of claim 1, wherein the biological fluid specimen collector is adapted for at least one of nasopharyngeal and oropharyngeal sampling.

4. The device of claim 1 further comprising a flexible application injector extending from an application tip of the cartridge nest, and wherein the biological fluid specimen collector is an absorbent material located at least partially around the application injector.

5. The device of claim 4, wherein the absorbent material is a sterile cotton swab.

6. The device of claim 1, wherein the biological fluid specimen collector is adapted for removal from the device and transported to a laboratory for analysis.

7. The device of claim 1 further comprising an integrated electronics subassembly configured for collecting vital signs information.

8. The device of claim 7, wherein the electronics subassembly comprises a temperature sensor for taking a patient's temperature.

9. The device of claim 8, wherein the temperature sensor is an infrared proximity temperature sensor.

10. A secure device for delivering medications, the device comprising:
a syringe body;
a cartridge nest;
a plunger actuator; and
an integrated electronics subassembly configured for collecting vital signs information.

11. The device of claim 10, wherein the electronics subassembly is adapted for interfacing with a peripheral sensor capable to detect and transmit the patient vital signs information data.

12. The device of claim 11, wherein the peripheral sensor is included in a wearable device.

13. The device of claim 10, wherein the electronics subassembly is adapted for notifying at least one of a patient, healthcare provider and administrator when at least one vital sign parameter meets a predetermined threshold.

14. The device of claim 10, wherein the electronics subassembly comprises a temperature sensor for taking a patient's temperature.

15. The device of claim 14, wherein the temperature sensor is an infrared proximity temperature sensor.

16. The device of claim 10, wherein the electronics subassembly is configured for transmitting a patient's collected vital signs information to a database for storage.

17. The device of claim 10 further comprising a biological fluid specimen collector.

18. The device of claim 17, wherein the biological fluid specimen collector is a nasal fluid specimen collector.
